(19) United States
(12) Patent Application Publication
(10) Pub. No.: US 2015/0313573 A1
(43) Pub. Date: Nov. 5, 2015

(54) OPHTHALMIC ELASTOGRAPHY

(71) Applicant: Ohio State Innovation Foundation, Columbus, OH (US)

(72) Inventors: Jun Liu, Columbus, OH (US); Hong Chen, Columbus, OH (US)

(21) Appl. No.: 14/701,996

(22) Filed: May 1, 2015

Related U.S. Application Data
(60) Provisional application No. 61/987,684, filed on May 2, 2014.

(51) Int. Cl. A61B 8/10; A61B 3/16

(52) U.S. Cl. CPC A61B 8/10; A61B 3/16

(57) ABSTRACT

This invention describes an ultrasound technique that maps out the mechanical properties of the cornea and the sclera to the intrinsic mechanical loadings in the eye. It helps identify the abnormally weaker or stiffer regions in the eye, to add functional information for early and definitive diagnosis of corneal diseases, surgical planning, prevention of surgical complications, as well as better interpretation of tonometric readings. This technique will allow a spatial mapping of the mechanical strains developed in the cornea or the sclera during ocular pulse or other intraocular pressure fluctuations. The envisioned use of this technique resembles the current clinical ophthalmic ultrasound in terms of the patient experience, but provides functional information about the eye tissue that is not available from current clinical ultrasound.

### CROSS REFERENCE TO RELATED APPLICATION

This application claims priority to U.S. Provisional Application No. 61/987,684 filed on May 2, 2014, which is incorporated herein by reference in its entirety.

### FIELD OF THE INVENTION

The present invention relates to methods and systems for determining the biomechanical properties of the eye. In particular, the present invention relates to in vivo mapping of the cornea and sclera biomechanical properties using high frequency ultrasound.

### BACKGROUND OF THE INVENTION

Structural information of the cornea such as central corneal thickness and topography is critical for disease diagnosis and surgical planning. However, it remains challenging to predict, for example, ectasia risk secondary to keratoconus and refractive surgery, even though detailed structural information has become available. Thus, structural data alone are often not sufficient to make informed clinical decisions without also having functional information, such as would be available from measures of the biomechanical properties of the cornea in relationship to the status or changes in structure. The present invention provides for a method and system to use ultrasound elastographic technique for in vivo imaging of corneal biomechanical responses to intrinsic physiological mechanical forces, i.e., the ocular pulse.

At the present there are no in vivo methods that can image spatially resolved mechanical properties of the cornea that are also independent of the intraocular pressure (IOP). The ultrasound elastography technique described in this invention harnesses the resolution and accuracy achieved by customized radiofrequency analyses of high frequency ultrasound data to map the deformation of the cornea during the loading/unloading cycles of the ocular pulse. From these measures, the biomechanical properties of the cornea independent of IOP can be estimated. The elastographic technique also leverages on its intrinsically higher sensitivity to a weaker response resulting in a larger and more easily measurable strain.

The methods and systems of the present invention allow for identification of the presence of biomechanical instability or weakening prior to the onset of clinical diseases. The methods and system of the present invention provide a cost-effective and quick evaluation of the biomechanical conditions of the cornea which will aid clinical diagnosis and decision-making, for example, in the prediction of ectasia risk prior to refractive surgery, the early and definitive diagnosis and staging of keratoconus, the interpretation of tonometry measurements in patients with abnormal corneas, and the therapeutic planning and monitoring of ectasia diseases such as corneal crosslinking. In addition, the methods and systems of the present invention provide a tool for studying ocular biomechanics in health as well as the course of many different types of ocular disease.

### SUMMARY

The methods and systems of the present invention provide for a method for determining the biomechanical properties of an eye comprising the steps of: (a) providing an ultrasound transducer in proximity to the eye or on the eye, wherein the ultrasound transducer has a center frequency ranging from about 50 MHz to about 100 MHz, or from about 50 MHz to about 80 MHz; (b) acquiring ultrasound data from at least one cross-section of at least one portion of the eye; (c) measuring ocular pulse and intraocular pressure: and, (d) processing the ultrasound data from step (b) with a speckle tracking algorithm to generate a strain profile of the cross-section as a function of time during the ocular pulse. The strain profile may include global and/or local strain magnitudes, and/or strain rates.

The methods and systems of the present invention also provide for a method of diagnosing eye disease or monitoring treatment effectiveness for eye disease, the method comprising the steps of: (a) providing an ultrasound transducer in proximity to the eye or on the eye, wherein the ultrasound transducer has a center frequency ranging from about 50 MHz to about 100 MHz, or from about 50 MHz to about 80 MHz; (b) acquiring ultrasound data from at least one cross-section of at least one portion of the eye; (c) measuring ocular pulse and intraocular pressure; (d) processing the ultrasound data from step (b) with a speckle tracking algorithm to generate a strain profile of the cross-section as a function of time during the ocular pulse; and, (e) comparing the strain profile to a standard, wherein, the standard provides the strain profile for normal subjects and/or patients having eye disease. A stiffness index may also be produced from the data. The eye disease can be keratoconus or glaucoma. The eye disease may be any eye-condition that can be treated with corneal crosslinking or a topical drug.

Methods for planning incision sites in an eye (e.g., prior to surgery) are also included. The steps for planning such incisions comprise: (a) providing an ultrasound transducer in proximity to the eye or on the eye, wherein the ultrasound transducer has a center frequency ranging from about 50 MHz to about 100 MHz, or from about 50 MHz to about 80 MHz; (b) acquiring ultrasound data from at least one cross-section of at least one portion of the eye; (c) measuring ocular pulse and intraocular pressure; (d) processing ultrasound data from step (b) with a speckle tracking algorithm to generate a strain profile of the cross-section as a function of time during the ocular pulse; and, (e) determining an optimum set of incision sites based on the strain profile. A stiffness index may also he produced. The incision can be made before or during corneal transplant for partial corneal transplant) or cataract surgery. The strain profile of either the cornea or sclera may he generated.

A method for predicting ectasia risk from refractive surgery is also encompassed by the invention. The methods comprises the steps of: (a) providing an ultrasound transducer in proximity to the eye or on the eye, wherein the ultrasound transducer has a center frequency ranging from about 50 MHz to about 100 MHz, or from about 50 MHz to about 80 MHz; (b) acquiring ultrasound data from at least one cross-section of at least one portion of the eye; (c) measuring ocular pulse and intraocular pressure; (d) processing the ultrasound data of step (b) with a speckle tracking algorithm to generate a strain profile and a stiffness index of the cross-section; and, (e) determining risk for ectasia risk based on the strain profile and stiffness-index. The refractive surgeries can be for correcting myopia (e.g., LASIK) or presbyopia.

Methods for predicting complications related to abnormal intraocular pressure elevations during intraocular injection treatments are encompassed and comprise the steps of: (a) providing an ultrasound transducer in proximity to the eye or on the eye, wherein the ultrasound, transducer has a center frequency ranging from about 50 MHz to about 100 MHz, or from, about 50 MHz to about 80 MHz; (b) acquiring ultrasound data from at least one cross-section of at least one portion of the eye; (c) measuring ocular pulse and intraocular pressure; (d) processing the ultrasound data, of step (b) with a speckle tracking algorithm to generate a strain profile and a stiffness index of the cross-section; and, (e) determining the risk for ocular-injection related complications based on the strain profile and stiffness index. Intraocular injection, treatments can be for treating macular degeneration (e.g., age-associated).

Also encompassed by the present invention are methods for correcting tonometric measurements of intraocular pressure. The method may comprise the steps of: (a) providing an ultrasound transducer in proximity to the eye or on the eye, wherein the ultrasound transducer has a center frequency ranging from about 50 Mite to about 100 MHz, or from about 50 MHz to about 80 MHz; (b) acquiring ultrasound data from at least one cross-section of at least one portion of the eye; (c) measuring ocular pulse and intraocular pressure; (d) processing the ultrasound data of step (b) with a speckle tracking algorithm to generate a strain profile and a stiffness index of the cross-section; and, (e) comparing the strain profile and the stiffness index to a standard, wherein the standard provides the strain profile and the stiffness index for normal subjects and/or patients having eye disease, and determining whether IOP is significantly under- or over-estimated by tonometric readings. The tonometric measurements may be from Goldmann Applanation Tonometry or Tonopen.

In the present methods, a stiffness index may also be generated for at least one portion of the eye. The portion of the eye may be a cornea or a sclera. The cornea or sclera can be divided into at least two layers. The layers can be the anterior, middle or posterior. Each layer can further be divided into at least two zones. The zones can be the nasal, central or temporal zones. A stiffness index and a heterogeneity score can be calculated, for each layer or zone. In the present methods, in step (d), the speckle tracking algorithm may read digitized radiofrequency scan lines of the ultrasound data to generate the strain profile.

The ocular pulse and intraocular pressure can be measured (step (e)) before, during or after application of the ultrasound to the eye (steps (a) and (b)).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flow chart of a speckle tracking algorithm.

FIG. 2 shows schematics of the overall system and process of an embodiment of the present methods and systems.

FIG. 3 shows an ultrasound probe covered with a cellulose membrane.

FIG. 4 shows the plots of Eq. 2 when “b” values are 0.05, 0.1 and 0.2. The strains at other IOPs are all calculated relative to the IOP of 5 mmHg.

FIG. 5 shows an example of five cycles of experimentally measured radial strains and the corresponding IOP.

FIG. 6 shows the strain-IOP plot based on the data in FIG. 4. Strain values are multiplied by 100 to increase the accuracy of the estimated slope “k.”

FIG. 7 shows an example of the predicted strain-IOP curve corresponding to the “b” value obtained from experiment.

### DESCRIPTION OF THE INVENTION

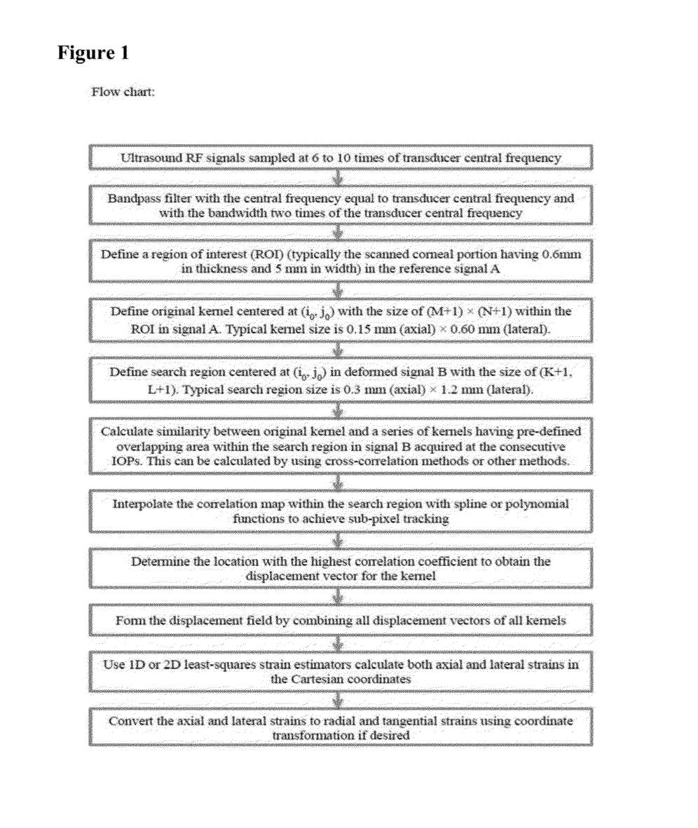

The methods and systems of the present invention generate an estimate of the average stiffness as well as the spatial mapping of the mechanical strains developed in the cornea or the sclera during ocular pulse or other intraocular pressure fluctuations. The methods and systems further provide functional information about the eye tissue that is not available from the current clinical ultrasound. The overall system schematic is shown in FIG. 1 and described in detail as follows.

In one embodiment, a patient under examination is either supine or sitting. The patient's head is kept still either by a chin-and-forehead rest in sitting or by a head immobilizer in supine. The patient is instructed to fixate on a spot along the visual axis using the non-measured eye to limit any eye motion during the measurements.

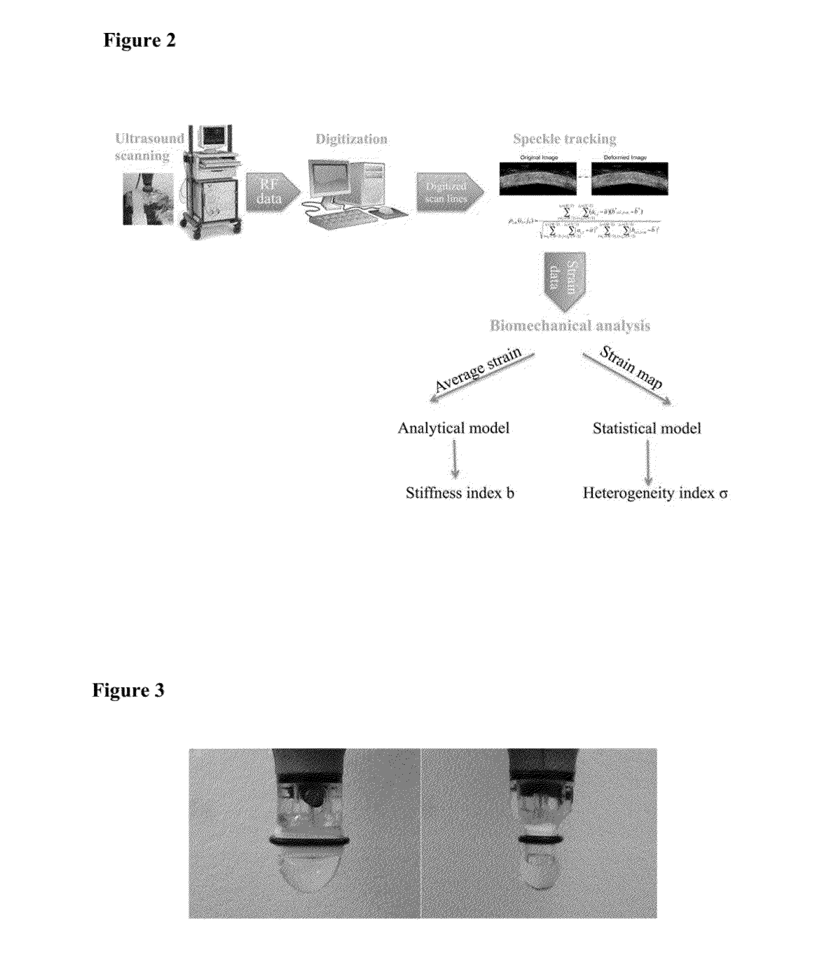

During measurements, a high-frequency ultrasound probe (or transducer) is placed by a precision positioning arm in the close proximity to the patient's eye. The probe, with a focused ultrasound transducer at the central frequency of, e.g., 55 MHz or higher, is immersed in distilled water enclosed by a cellulose membrane. For example, the probe may be the same as in the current B-mode ophthalmic ultrasound (as shown in FIG. 2). The cellulose membrane is very compliant and will conform to the shape of the eye surface upon contact. A layer of eye lubricating gel (e.g., GenTeal) is applied on the cellulose membrane to couple between the probe and the surface of the eye.

The subject's eye can be closed or open. When the eye is closed, the ultrasound transducer may or may not be in contact with the closed eyelid. In one embodiment, the present method is carried out through a closed eyelid. In one embodiment, a sufficient amount of ultrasound transmission gel can be applied to the subject's closed eyelid so that the ultrasound transducer is not in direct contact with the eyelid. When the eye is open, the ultrasound transducer may or may not be in contact with the eye. In another embodiment, the present method is carried out on an open eye.

When the ultrasound transducer is positioned in proximity to the eye, the transducer may be placed on the closed eyelid, may be in contact with ultrasound transmission gel that has been applied on the closed eyelid, or may be placed in close distance to an open eye.

The center frequency of the ultrasound transducer of the present method and system may range from about 50 MHz to about 100 MHz, from about 50 MHz to about 80 MHz, from about 55 MHz to about 90 MHz, from about 55 MHz to about 80 MHz, from about 60 MHz to about 70 MHz, from about 50 MHz to about 60 MHz, from about 70 MHz to about 100 MHz, or may be 50 MHz or higher, 55 MHz or higher, 60 MHz or higher, or 70 MHz or higher. The ultrasound probe may be positioned in proximity to the eye or on the eye.

The probe is positioned first to scan the nasal-temporal cross-section of the cornea or sclera. High frequency ultrasound analogue radiofrequency data are acquired for a few seconds during fixation at a frame rate of ranging from about 5 to about 30 frames per second, using a high sampling rate digitizer (over 500 M samples per second) controlled for example by a LabVIEW or C++ interface. The probe can be repositioned to scan any other cross-sectional plane of interest if so desired. At least three repeats are obtained, for each plane.

Sampling rates of ultrasound measurement may vary. For example, the sampling frequency may range from about 5 to about 50 frames per second, from about 5 to about 40 frames per second, from about 5 to about 30 frames per second, from about 10 to about 20 frames per second, from about 15 to about 25 frames per second, from about 15 to about 30 frames per second, from about 20 to about 30 frames per second, about 10 frames per second, about 15 frames per second, about 20 frames per second, about 25 frames per second, or about 30 frames per second. The speed of digitization of the A-lines may vary depending on the center frequency of the ultrasound transducer, and may range from about 200 MHz to about 1 GHz, from about 300 MHz to about 900 MHz, from about 400 MHz to about 800 MHz, from about 500 MHz to about 700 MHz, from about 200 MHz to about 500 MHz, or from about 300 MHz to about 600 MHz. In one embodiment, in order to improve the sampling frequency for each cardiac cycle, a retrospective ECG gating technique can be used to combine the scans from multiple heart beats into one cycle, and thus achieve higher temporal scanning rate and improve strain tracking accuracy. Lao J, Konofagou E E. High-frame rate, full-view myocardial, elastography with automated contour tracking in murine left ventricles in vivo. IEEE Trans Ultrason Ferroelectr Freq Control. 2008; 55(1):240-8.

In another embodiment, the present methods and systems use optical coherence tomography (OCT). For example, data used for generation of stiffness index and strain profile from the eye may be acquired using OCT. Wang et al. Optics: 39(1):41 (2014); Ford et al. J. Biomedical Optics 16(1):016005 (2011).

Either before, after or during ultrasound scanning, the ocular pulse as well as the intraocular pressure (IOP) of the patient is measured by a device, including, but not limited to, the Dynamic Contour Tonometry, which is an FDA approved clinical device, to obtain the profile of the intraocular pressure (baseline and ocular pulse) (see, e.g., Punjab et al. Dynamic Contour Tonometry: Principle and Use. Clin. Exp. Opthalmol 34(9): 837 (2006)). Other methods for measuring ocular pulse may also be used. The corneal thickness and curvature maps may also be obtained using existing standard ophthalmic instruments. The IOP fluctuations could be ocular pulse, IOP changes due to postural change (e.g., sitting or supine), IOP changes due to fluid intake (e.g., water drinking), or IOP changes due to valsalva maneuver (e.g., holding breath).

The acquired ultrasound radiofrequency data are processed by using a speckle tracking algorithm. This algorithm reads the digitized radiofrequency scan lines and outputs the distributive strain data of a given cross-section of at least one portion of the eye as a function of time during the ocular pulse. The average strain within a cross-section is combined with the data of intraocular pressure and ocular pulse to derive the biomechanical properties (e.g., the stiffness index) of the cornea and the sclera using an analytical model (as described herein including Example 1).

Ultrasound strain mapping is achieved using speckle tracking techniques applied to ultrasound signals acquired at both undeformed and deformed states. Ultrasound speckle tracking estimates tissue displacements using either cross-correlation algorithms (O'Donnell et al., Internal Displacement and Strain Imaging Using Ultrasonic Speckle Tracking, Ieee Transactions on Ultrasonics Ferroelectrics and Frequency Control, 1994; 41:314-325. Ophir et al., Elastography: a quantitative method for imaging the elasticity of biological tissues. Ultrasonic imaging 1991; 13:111-134), sum of absolute differences (Bohs et al., A Novel Method for Angle Independent Ultrasonic-Imaging of Blood-Flow and Tissue Motion, Ieee Transactions on Biomedical Engineering, 1991; 38:280-286), or optic flow methods (Angelini. et al., Review of myocardial motion estimation methods from optical flow tracking on ultrasound data, 2006, 28th Annual International Conference of the Ieee Engineering in Medicine and Biology Society, Vols 1-15 2006; 6337-6340). The strain is calculated as the spatial gradient of the displacement field. Kallel et al., A least-squares strain estimator for elastography. Ultrasonic Imaging 1997; 19:195-208. Tissue deformation needs to be induced either by intrinsic forces or external forces. Tang et al. J. Biomechanical Engineering 134(9):091007 (2012).

The ultrasound data, e.g., the ultrasound RF (radiofrequency) signals, may be first filtered using a band-pass filter with a bandwidth two times of the transducer central frequency (e.g., 100 MHz or 110 MHz, etc.) centered at the transducer central frequency (e.g., 50 MHz or 55 MHz, etc.) to remove noise. A correlation-based speckle tracking algorithm (Cohn et al., An elasticity microscope .1. Methods. Ieee Transactions on Ultrasonics Ferroelectrics and Frequency Control 1997; 44:1304-1319) is applied to the RF signals obtained at two consecutive pressure levels. Briefly, a kernel in the original signal A centered at (i₀, j₀) was compared with a series of kernels in the deformed signal B near the neighborhood of the original kernel. The correlation coefficient between the original, kernel in signal A and the kernel in signal B centered at (i₀+l, j₀+m) is calculated as follows (Equation 1):

$${ρ}_{l , m} ( {i}_{0} , {j}_{0} ) = \frac{{\sum }_{i = {i}_{0} - ( M / 2 )}^{{i}_{0} + ( M / 2 )} {\sum }_{j = {j}_{0} - ( N / 2 )}^{{j}_{0} + ( N / 2 )} ( {a}_{i , j} - \overline{a} ) ( {b}_{i + l , j + m}^{⋆} - {\overline{b}}^{⋆} )}{\sqrt{{\sum }_{i = {i}_{0} - ( M / 2 )}^{{i}_{0} + ( M / 2 )} {\sum }_{j = {j}_{0} - ( N / 2 )}^{{j}_{0} + ( N / 2 )} {\vert {a}_{i , j} - \overline{a} \vert}^{2} {\sum }_{i = {i}_{0} - ( M / 2 )}^{{i}_{0} + ( M / 2 )} {\sum }_{j = {j}_{0} - ( N / 2 )}^{{j}_{0} + ( N / 2 )} {\vert {b}_{i + l , j + m} - \overline{b} \vert}^{2}}} \qquad ( 1 )$$

where ā and b̅ are the values in signal A and B, and the ā and b̅ are the average values of the corresponding kernels. The size of the kernel was (M+1)×(N+1) data points, corresponding to a region of (M+1)×(N+1) pixels in the ultrasound image.

The correlation coefficients are calculated for a search region around (i₀, j₀) in signal B by varying l and m, and interpolated using a spline function to achieve sub-pixel tracking. The location with the largest correlation coefficient magnitude is used to determine the displacement vector. The displacement fields are accumulated over consecutive pressure levels. A least-square strain estimator is used to calculate the strains in both the axial (along the ultrasound beam) and lateral (perpendicular to ultrasound beam) directions, Kallel et al., A least-squares strain estimator for elastography, Ultrasonic Imaging 1997; 19:195-208.

Strains along any arbitrary direction (e.g., radial and tangential strains) can be calculated from the displacement field using coordinate transform. Average strains (either axial and lateral; or tangential and radial) are obtained within a region of interest. The strain fields may be smoothed following the typical procedures used in the ultrasound elastography field. O'Donnell et al., Internal Displacement and Strain Imaging Using Ultrasonic Speckle Tracking, Ieee Transactions on Ultrasonics Ferroelectrics and Frequency Control, 1994; 41:314-325. Cohn et al., An elasticity microscope .1. Methods. Ieee Transactions on Ultrasonics Ferroelectrics and Frequency Control 1997; 44:1304-1319.

A flow chart incorporating the speckle tracking algorithm is in FIG. 1.

The distributive strain data are analyzed to evaluate the degree of heterogeneity in the scanned regions. For example, the cornea or sclera is divided into three layers (e.g., anterior, middle, and posterior) and the average strains of each layer are compared to examine whether there are through-thickness heterogeneity. Likewise, the scanned region can be divided into three zones (e.g., nasal, central, and temporal) to evaluate whether there are large regional variances. A heterogeneity score can be assigned to indicate the extent of strain heterogeneity.

The biomechanical data are combined with structural evaluations such as corneal thickness profile and curvature maps to develop clinical scoring systems for ectasia risk for refractive surgery candidates and keratocoous patients/suspects. The overall stiffness index of the cornea can be used to calibrate the readings of intraocular pressure from Goldmann Applanation Tonometry.

FIG. 2 shows the schematics of the overall system and process.

At least two types of strains are available for analysis. Each strain can be of distinct diagnostic values. These strains include, but are not limited to, tensile strains that describe how stretchable the cornea is along the in-plane direction; and the compressive strains that describe how compressible the cornea is through thickness. Both strains may be simultaneously or sequentially computed by the ultrasound speckle tracking algorithm.

FIG. 3 shows the ultrasound probe covered with a cellulose membrane.

The methods and systems of the present invention may be integrated with commercially existing ophthalmic ultrasound B-mode imaging systems through an “RF out port”. For example, AVISO produces a high-frequency A/B scan ultrasound platform that allows for 50 MHZ scanning. The “RF out” port can be used to make the uncompressed, unsampled radiofrequency data accessible to the present methods and systems for further data processing to compute the strain data. In one embodiment, the ophthalmic elastography methods and system of the present invention may be a digitizing card and a computer with the data analysts software incorporated therein.

The methods and systems of the present invention has important clinical Implications. For example, keratoconus is a non-inflammatory, degenerative corneal disease that has an incidence of 1/2000. It often starts at puberty and progresses into corneal ectasia (i.e., a conical protrusion) that severely distorts vision. Early detection of keratoconus is important for disease management and prognosis but definitive diagnosis and staging of keratoconus is currently difficult even with advanced structural evaluations. A related situation is the screening for refractive surgery candidates where subclinical keratoconus or intrinsically weaker corneas likely have contributed to the incidence of postsurgical ectasia. The present noninvasive elastographic method adds an evaluation of the cornea's mechanical condition for more effective therapeutic planning of keratoconus and better prevention of ectasia secondary to keratoconus or refractive surgery.

Beyond ectatic diseases, the availability of corneal biomechanical evaluation could also help the clinical care for glaucoma suspects and patients in at least two important aspects. First, the proposed methods and systems will allow for a more accurate interpretation of tonometric readings of IOP, which is a primary risk factor for glaucoma. A mechanically abnormal cornea could result in significant over or underestimates of IOP and in vivo measurement of corneal biomechanics is needed for better interpreting IOP readings in these patients. Secondly, corneal and scleral biomechanical properties, as well as their change due to age and disease, are factors influencing glaucoma risk. The present methods and systems provide a clinical as well as research tool to quantify these properties in vivo and help better understand their roles in glaucoma.

Third, the measurement of cornea and scleral biomechanical properties can help surgeons make better decisions in terms of the incision sites and cornea orientation during corneal transplant and cataract surgeries. One major complication of these surgeries is postsurgical astigmatism. The preexisting regional biomechanical profile may help identify patients with high risk of astigmatism. In addition, knowledge of the regional comparisons of biomechanics can help tire surgeon select the area of incision to avoid creating biomechanical heterogeneities. Tejedor et al., Am. J. Ophthalmol. May; 139(5):767-6 (2005).

Three-dimensional (3D) images of the cornea, representing quasi-volume metric measurements of the cornea, may be developed using the methods and systems of the present invention. Fenster et al., Three-dimensional ultrasound imaging. Phys. Med. Biol. 46 R67-99 (2001). For example, the cornea is scanned along at least six meridians or at least twelve meridians (for example, one at each clock hour), and a three dimensional image constructed using standard techniques. In one embodiment, the 2D strain data, from each cross-sectional scan are interpolated to obtain a quasi-volumetric representation of the cornea.

### EXAMPLES

The following Examples are not to be construed as limiting.

### Example 1

### Analytical Model For Deriving the Biomechanical Index From Strain Measurements

Based on the analysis of inflation tests of corneas, an analytical model is used to fit the strain-IOP curve. The strain-IOP curve passes through the point (5 mmHg, 0%) because an IOP of 5 mmHg is used as the reference pressure (i.e., 0% strain). Equation 2 (Eq. 2) shows the relationship between IOP and the radial strain, which can be accurately measured during ocular pulse.

strain=b*IOP⁻¹−b*5⁻¹ Eq. 2

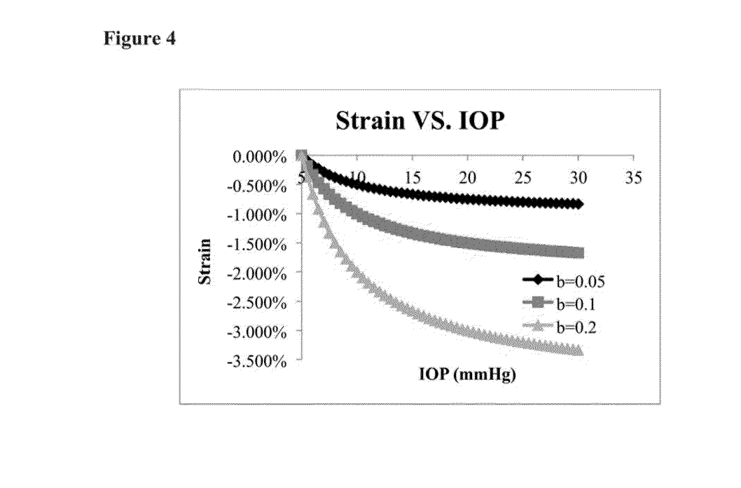

In this model there is only one unknown parameter (“b”), which determines the overall shape of the strain-IOP curve, to predict the strain response of the cornea at a given IOP. As shown in FIG. 4, a smaller “b” value gives a shallower curve, indicating a stiffer cornea, and a larger “b” value gives a deeper curve, indicating a more compliant cornea. Therefore, “b” can be used as a stillness index obtained from the strain-IOP response.

FIG. 4 shows the plots of Eq. 2 when “b” values are 0.05, 0.1 and 0.2. The strains at other IOPs are all calculated relative to the IOP of 5 mmHg.

During ocular pulse, the strains corresponding to a small fluctuation of the IOP are recorded using the high frequency ultrasound elastography technique.

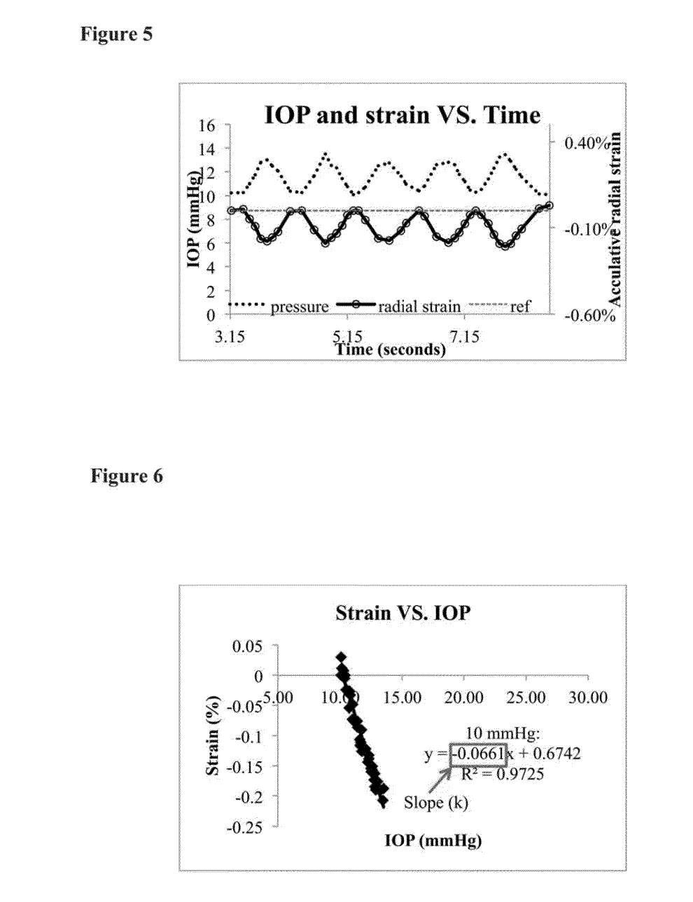

FIG. 5 shows an example of the cyclic ocular pulse induced in an ex vivo eye and the corresponding cyclic corneal strains measured from ultrasound. The strain and IOP data are then plotted to generate an estimate of the slope “k” that represents the slope of the strain-IOP curve at the point of the baseline IOP.

FIG. 6 is the strain-IOP plot based on the data in FIG. 4. Strain values are multiplied by 100 to increase the accuracy of the estimated slope “k.”

The in vivo strain measurement cannot be directly applied in Eq. 2 to calculate the “b” value because the reference pressure (i.e., the baseline pressure) is typically not at 5 mmHg and varying from subject to subject. For data shown in FIG. 5, strains are measured using the reference image acquired at the baseline IOP of 10 mmHg. The slope k at any given pressure in a nonlinear strain-IOP curve is independent of the reference IOP. Since the overall range of the ocular pulse (the ocular pulse amplitude) is usually vey small, around 3 mmHg in average, the slope k corresponding to the baseline IOP can be obtained by applying a linear fitting to the strain-IOP data from in-vivo measurements during the ocular pulse. The “b” value is then calculated by applying slope k (estimated) and the baseline IOP (measured) into the Eq. 3, which is from the derivative of Eq. 2 with respect to IOP.

b=−k·IOP_{base}² Eq. 3

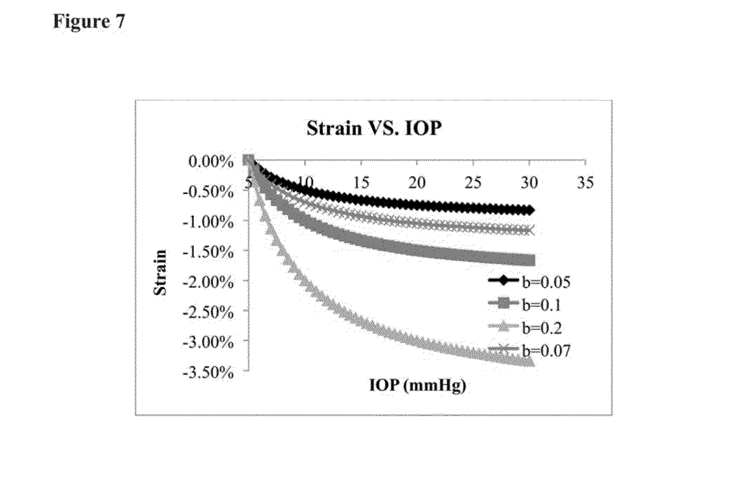

The resultant “b” value is used as the stiffness index to compare the overall stiffness between different eyes. In the example shown in FIG. 5, the estimated b is 0.07, generating a strain-IOP curve shown in FIG. 7.

FIG. 7 shows an example of the predicted strain-IOP curve corresponding to the “b” value obtained from experiment.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

We claim:

1. A method for determining biomechanical properties of an eye comprising the steps of:
(a) providing an ultrasound transducer in proximity to the eye or on the eye, wherein the ultrasound transducer has a center frequency of about 50 MHz to about 80 MHz;
(b) acquiring ultrasound data from at least one cross-section of at least one portion of the eye;
(c) measuring ocular pulse and intraocular pressure; and,
(d) processing the ultrasound data from step (b) with a speckle tracking algorithm to generate a strain profile of the cross-section as a function of time during the ocular pulse.

2. The method of claim 1, wherein in step (d) the speckle tracking algorithm reads digitized radiofrequency scan lines of the ultrasound data to generate the strain profile.

3. The method of claim 1 wherein in step (d) a stiffness index is generated.

4. The method of claim l, wherein the portion of the eye is a cornea or a sclera.

5. The method of claim 4, wherein the cornea or sclera is divided into at least two Layers.

6. The method of claim 5, wherein the layers are anterior, middle or posterior.

7. The method of claim 5, wherein the layer is divided into at least two zones.

8. The method of claim 7, wherein the zones are a nasal, a central or a temporal zone.

9. The method of claim 8, wherein a heterogeneity score is calculated for each zone.

10. The method of claim 1, wherein step (c) is carried out before, during or after steps (a) and (b).

11. The method of claim 3, wherein the strain profile and stiffness index are used to predict ectasia risk for refractive surgery.

12. The method of claim 11, wherein the refractive surgery comprises a surgery for correcting myopia or presbyopia.

13. The method of claim 3, wherein the strain profile and stiffness index are used to determine and/or monitor efficacy of treatment for ectasia.

14. The method of claim 3, wherein the strain profile and stiffness index are used to predict risk of complications related to abnormal intraocular pressure elevations during an intraocular injection.

15. The method of claim 3, wherein the strain profile and stiffness index are used to correct tonometric measurements of intraocular pressure, where the strain profile and stiffness index are compared to a standard, wherein the standard provides the strain profile and stiffness index for normal subjects and/or patients having eye disease.

16. A method of diagnosing eye disease or monitoring treatment, effectiveness for eye disease comprising the steps of:
(a) providing an ultrasound transducer in proximity to the eye or on the eye, wherein the ultrasound transducer has a center frequency of about 50 MHz to about 80 MHz;
(b) acquiring ultrasound data from at least one cross-section of at least one portion of the eye;
(c) measuring ocular pulse and intraocular pressure;
(d) processing the ultrasound data from step (b) with a speckle tracking algorithm to generate a strain profile of the cross-section as a function of time during the ocular pulse; and,
(e) comparing the strain profile to a standard, wherein the standard provides the strain profile for normal subjects and/or patients having eye disease.

17. The method of claim 16, wherein a stiffness index is produced.

18. The method of claim 16, wherein the eye disease is keratoconus or glaucoma.

19. A method of planning incision sites in an eye prior to surgery, comprising the steps of:
(a) providing an ultrasound transducer in proximity to the eye or on the eye, wherein the ultrasound transducer has a center frequency of about 50 MHz to about 80 MHz;
(b) acquiring ultrasound data from at least one cross-section of at least one portion of the eye;
(c) measuring ocular pulse and intraocular pressure;
(d) processing the ultrasound data from step (b) with a speckle tracking algorithm to generate a strain profile of the cross-section as a function of time during the ocular pulse; and,
(e) determining an optimum set of incision sites based on the strain profile.

20. The method of claim 19, wherein a stiffness index is produced.

21. The method of claim 20, wherein the incision is made during corneal transplant or cataract surgery.

22. The method of claim 19, wherein the portion of the eye is a cornea or a sclera.
